(19) United States
(12) Patent Application Publication
(10) Pub. No.: US 2022/0054399 A1
(43) Pub. Date: Feb. 24, 2022

(54) MASKING SPRAY

(71) Applicant: GILLMOOER LLC, Centennial, CO (US)

(72) Inventors: Kimbra Mooers, Centennial, CO (US); Rachele Gillmar, San Mateo, CA (US); Nick Gillmar, San Mateo, CA (US); John Mooers, Centennial, CO (US)

(73) Assignee: GILLMOOER LLC, Centennial, CO (US)

(21) Appl. No.: 17/410,538

(22) Filed: Aug. 24, 2021

Related U.S. Application Data
(60) Provisional application No. 63/069,645, filed on Aug. 24, 2020.

(51) Int. Cl. A61K 8/9789; A61K 8/92; A61K 8/49; A61K 8/66; A61Q 19/00

(52) U.S. Cl. CPC A61K 8/9789; A61K 8/922; A61K 2800/87; A61K 8/66; A61Q 19/00; A61K 8/498

(57) ABSTRACT

One or more techniques and/or systems are disclosed for a composition or formulation that can be used as a surface applied treatment for reducing odors, enhancing a user experience with pleasant scents, and mitigating skin irritation for items worn close to a user's skin. The composition can comprise a natural astringent, such as witch hazel. Further, the composition can comprise an essential oil, such as lavender, mint, tea tree, grapefruit, ylang ylang, eucalyptus, and vanilla, along with distilled water. These ingredient can be readily mixed together, and packaged in an appropriate container, such as a spray bottle. The composition can be applied to a target surface, such as the inside of a mask, by spraying applying the composition.

### CROSS-REFERENCE TO RELATED APPLICATIONS

This application claims priority to U.S. Provisional Patent Application Ser. No. 63/069,645, entitled GILLMOOER LLC MASKING SPRAY, filed Aug. 24, 2020, which is incorporated herein by reference.

### BACKGROUND

Face masks or masks, sporting equipment, diaper changing mats, vehicle upholstery are components that are often made from many different materials, some of which include: paper, polypropylene, cotton, polyester, paper-like woven material, plastic, and many others. These components and materials can become highly charged with odors after use, especially continued, long-term use. These odors can be off-putting, and may also have an unpleasant effect on the user/wearer. Some existing remedies for mask odors use sprays that focus on diverting foul smells by wearing the mask. Another existing remedy involves the use of hydrogen peroxide or other irritating chemical oxidants in combination with odor controlling agents. Although the use of various oxidants may fight certain microbials, the use of a skin irritant can be problematic when used close to the skin for sustained periods of time, potentially exacerbating the skin irritation beyond what it would have been without the use of the skin-irritating solution. What is needed is a product with the purpose to refresh an object not only by making it smell better (by masking and/or reducing odors), but also refreshing an object(s) that comes in contact with the skin, such as a mask, gloves, sporting equipment, diaper changing mats, vehicle upholstery, watch bands, jewelry, clothing, shoes and many others as well as reduce mask-acne and other skin irritations; and objects for animals, such as dog and cat collars, saddles, and similar items that come into contact with animals.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

One or more techniques and systems are described herein for a surface applied composition that can be used to reduce malodors and also provide pleasant scents to enhance and promote the use of a personal item. Further, the composition can be configured to help mitigate skin irritation when applied to personal items that may lie against a user's skin. A natural astringent mixed with an essential oil, possibly other scents, along with distilled water can be used for the composition.

In one implementation a composition, a method of making a composition, and a method of using a composition, the composition can comprise a natural astringent comprising witch hazel in an amount sufficient to provide an astringent action to skin. Further the composition can comprise a natural scent made from an essential oil component comprising at least one essential oil selected from the group consisting of: lavender, mint, tea tree, grapefruit, ylang ylang, eucalyptus, and vanilla. Additionally, the composition can comprise a diluent comprising distilled water.

To the accomplishment of the foregoing and related ends, the following description set forth certain illustrative aspects and implementations. These are indicative of but a few of the various ways in which one or more aspects may be employed. Other aspects, advantages and novel features of the disclosure will become apparent from the following detailed description when considered in conjunction with the claims.

### DETAILED DESCRIPTION

The claimed subject matter is now described, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

As described herein, a composition of matter in the form of a fluid that is applied by coating (e.g., spraying) the intended area for use, such as the inside of a mask worn to mitigate airborne exposure to microbials, a yoga mat, or other components or locations to reduce odor, generate a pleasant aroma, and mitigate exposure to irritants. For example, the purpose (e.g., or at least a main purpose) is not to divert the smell by wearing the mask (e.g., or coming in close contact with the item to be refreshed), but rather to create a refreshing scent that enhances the experience of wearing a mask (e.g., or other use item), while having essential oils and a natural astringent to mitigate skin irritation and “mask acne”. Cumulatively, the result is a more pleasant experience using the items.

In one implementation, the formulation can be described as a masking spray, comprising a product that is formulated with a unique blend of witch hazel, distilled water, and one or more essential oils to help refresh products, such as masks (e.g., which may be worn due to airborne pandemics), sporting equipment, diaper changing mats, vehicle upholstery, in a plurality of ways. Masks and other target components are often made from many different materials, including, but not limited to, paper, polypropylene, cotton, polyester, paper-like woven material, plastic, and many others.

In some implementations, the masking spray described herein, when utilized on “masks,” can significantly reduce lingering orders, and also help protect against skin irritations that may result from long-term use of a mask, such as what is termed as “Mask-Acne” or “Maskne”. The formulated product can comprise a blend of one or more of the essential oils in that may create aromas that can help alleviate lingering orders, and improve the likelihood of users wanting to wear a mask, and keeping it on. As an example, some of the essential oils and other scents can include, Lavender, Mint and even Bubble Gum to create a pleasant experience. As an example, the use of the essential oil comprising formulation, when applied to “masks” or other item, can reduce malodors, mitigate lingering odors with the essential oils, and a natural astringent portion may help address skin irritation issues, such as mask acne.

In one implementation, the formulation comprises witch hazel, which is used as a natural astringent that has anti-inflammatory and antiviral properties, and may be useful in fighting acne and other skin conditions. Other astringents have been used, but they often produce other side effects, such as alcohol in hand sanitizers which may not be recommended to inhale, and may not provide a please and refreshing odor eliminating affect. Further, some sprays may include essential oils to provide a pleasant scent, but they do not offer the added benefit of a natural astringent, such as witch hazel, to keep mask-acne at bay as well as other skin irritations caused by wearing a mask or other

### How to Make it:

The main ingredients of the formulation described herein include: distilled water in an amount sufficient to make up the bulk of the product; witch hazel in a quantity sufficient to provide a natural astringent action when applied to a component, depending on the target use; one or more essential oils selected from the group consisting of: lavender, mint, tea tree, grapefruit, ylang ylang, eucalyptus, and vanilla. In some implementations, more than one essential oil may be used from the group above. Further, additional essential oils, not listed above, may be added to the group described. Additionally, one or more natural antihistamines may be used in some implementation, such as stinging nettle, quercetins, bromelain, and butterbur.

The ingredient may be mixed together in any appropriate process that results in a formulation comprising the ingredients ready for packaging. The resulting formulation can be packaged in a container appropriate for the target use, such as a bottle, spray bottle, jar with an application tool, etc.

In some other implementations, additional or different scents may be added. As an example, where parents may have a difficult time making their children wear masks, the essential oils and other scents or “flavors” can be formulated to improve mask wearing in children, while also helping with mask acne by including the natural astringent. As an example, scents can include “bubble gum”, vanilla, orange, and more. In one example, a scent of the formula can comprise a bubble gum “flavor” mixed with grapefruit essential oil, Ylang Ylang essential oil and eucalyptus essential oil.

### How to Use:

In one implementation of a masking spray, a standard two ounce spray bottle can be filled with the formulation described herein, although other sizes can be employed. To use, the user can simply spray the target area (mask, yoga mat, sporting equipment, or other similar appropriate surface) with the masking spray. As a result, the user will enjoy the benefits of the natural astringent and the natural qualities of essential oils, resulting in a more enjoyable smelling mask, as well as relief from mask-acne, for example.

In some implementations, the surface treatment composition can be left to dry on the treated or target surface before the item is used by the user. In other implementations, the personal use item may be used immediately after application. As an example, the target surface can comprise several masks to which the composition is applied, and the masks can be later used by different users. In other examples, a user may carry a personal-sized container (e.g., a spray bottle, such as a two ounce spray bottle), and apply to the composition to the personal use item (e.g., mask) as needed or desired.

The word “exemplary” is used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, At least one of A and B and/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of

Also, although the disclosure has been shown and described with respect to one or more implementations, equivalent alterations and modifications will occur to others skilled in the art based upon a reading and understanding of this specification and the claims. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure. In addition, while a particular feature of the disclosure may have been disclosed with respect to only one of several implementations, such feature may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

The implementations have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

What is claimed is:

1. A composition of a surface applied treatment for a personal use item, comprising:
a natural astringent comprising witch hazel in an amount sufficient to provide an astringent action to skin;
a natural scent made from an essential oil component comprising at least one essential oil selected from the group consisting of: lavender, mint, tea tree, grapefruit, ylang ylang, eucalyptus, and vanilla; and
a diluent comprising distilled water.

2. The composition of claim 1, comprising another scent comprising: vanilla, bubble gum, orange, lemon, grape, root beer, chocolate, and/or lime.

3. The composition of claim 1, disposed in a spray bottle.

4. The composition of claim 1, disposed in a two ounce spray bottle.

5. The composition of claim 1, comprising a natural antihistamine comprising one or more of: stinging nettle, quercetins, bromelain, and butterbur.

6. A method for making composition of a surface applied treatment for a personal use item, the method comprising mixing together:
a natural astringent comprising witch hazel in an amount sufficient to provide an astringent action to skin;
a natural scent made from an essential oil component comprising at least one essential oil selected from the group consisting of: lavender, mint, tea tree, grapefruit, ylang ylang, eucalyptus, and vanilla; and
a diluent comprising distilled water.

7. The method of claim 6, comprising disposing the resulting composition in a spray bottle.

8. The method of claim 6, comprising disposing the resulting composition in a two ounce spray bottle.

9. The method of claim 6, comprising mixing another scent into the composition, the scent comprising: vanilla, bubble gum, orange, lemon, grape, root beer, chocolate, and/or lime.

10. The method of claim 6, comprising mixing a natural antihistamine to the composition, the natural antihistamine comprising one or more of: stinging nettle, quercetins, bromelain, and butterbur.

11. A method of applying a surface applied treatment composition to a personal use item, the method comprising spraying the composition to a target surface of the personal item wherein the composition comprises:
a natural astringent comprising witch hazel in an amount sufficient to provide an astringent action to skin;
a natural scent made from an essential oil component comprising at least one essential oil selected from the group consisting of: lavender, mint, tea tree, grapefruit, ylang ylang, eucalyptus, and vanilla; and
a diluent comprising distilled water.

12. The method of claim 11, applying the surface applied treatment composition to the personal use item, wherein the composition comprises another scent comprising: vanilla, bubble gum, orange, lemon, grape, root beer, chocolate, and/or lime.

13. The composition of claim 11, applying the surface applied treatment composition to the personal use item, wherein the composition is disposed in a spray bottle.

14. The composition of claim 11, applying the surface applied treatment composition to the personal use item, wherein the composition is disposed in a two ounce spray bottle.

15. The composition of claim 11, applying the surface applied treatment composition to the personal use item, wherein the composition comprises a natural antihistamine comprises one or more of: stinging nettle, quercetins, bromelain, and butterbur.
